(19) United States
(12) Patent Application Publication
(10) Pub. No.: US 2014/0228226 A1
(43) Pub. Date: Aug. 14, 2014

(54) METHOD AND SYSTEM FOR DETERMINING CHROMOSOME ANEUPLOIDY OF SINGLE CELL

(71) Applicants: Xuyang Yin, Shenzhen (CN); Chunlei Zhang, Shenzhen (CN); Yong Qiu, Shenzhen (CN); Shengpei Chen, Shenzhen (CN); Hui Jiang, Shenzhen (CN); Jun Wang, Shenzhen (CN); Jian Wang, Shenzhen (CN)

(72) Inventors: Xuyang Yin, Shenzhen (CN); Chunlei Zhang, Shenzhen (CN); Yong Qiu, Shenzhen (CN); Shengpei Chen, Shenzhen (CN); Hui Jiang, Shenzhen (CN); Jun Wang, Shenzhen (CN); Jian Wang, Shenzhen (CN)

(73) Assignee: BGI HEALTH SERVICE CO., LTD., SHENZHEN (CN)

(21) Appl. No.: 14/346,067

(22) PCT Filed: Sep. 21, 2011
(86) PCT No.: PCT/CN2011/079972

§ 371 Date: Mar. 20, 2014

(51) Int. Cl. G06F 19/22; C12Q 1/68

(52) U.S. Cl. CPC G06F 19/22; C12Q 1/6883
USPC 506/2; 506/38; 506/36; 702/19

(57) ABSTRACT

Disclosed is a method for determining the chromosome aneuploidy of a single cell and a system for determining the chromosome aneuploidy of a single cell. Among them, the method for determining the chromosome aneuploidy of a single cell according to the embodiments of the present invention comprises: the whole genome of the single cell is sequenced to obtain a first sequencing result; the total number of sequencing data from the first sequencing result is counted, obtaining a value L; the number of sequencing data of a first chromosome from the first sequencing result is counted, obtaining a value M; a first parameter is determined based on the value L and the value M; and it is determined whether or not the single cell has aneuploidy in respect of the first chromosome based on the difference between the first parameter and a predetermined control parameter.

### FIELD

The present disclosure relates to biomedical field, and particularly to a method and a system of determining a chromosome aneuploidy of a single cell.

### BACKGROUND

Chromosome aneuploidy closely relates to some human genetic diseases. Down syndrome is one of the most common chromosome aneuploidy, occurring in 1 in 1000, which results from having an extra chromosome 21. Trisomy 13 syndrome and trisomy 18 syndrome are caused respectively by having an extra chromosome 13 or an extra chromosome 18, with an emergence of miscarriage, and etc. Autosome aneuploidy is another important reason resulting in pregnancy failure and miscarriage. Sex chromosome abnormalities may cause abnormal sexual development. An individual in which males have an extra X chromosome (47, XXY) is Klinefelter syndrome. Turner syndrome, also known as congenital ovarian dysgenesis syndrome, is caused by being absent of an entire sex chromosome with a karyotype 45, X.

However, the method of detecting the chromosome aneuploidy still needs to be improved.

### SUMMARY

The present disclosure directs to solve at least one of the problems existing in the prior art. For this purpose, one aspect of the present disclosure provides a method which may effectively determine a chromosome aneuploidy of a single cell. Another aspect of the present disclosure provides a system which may effectively conduct the above method to determine the chromosome aneuploidy of the single cell.

The method of determine the chromosome aneuploidy of the single cell according to embodiments of the present disclosure may comprise following steps: sequencing a whole genome of the single cell to obtain a first sequencing result; counting the total number of sequencing data which can be aligned to a reference genome (also regarded as “a known genome”) in the first sequencing result, to obtain a value L; counting the number of sequencing data which can be aligned to a first chromosome in the reference genome in the first sequencing result, to obtain a value M; determining a first parameter based on the value L and the value M; determining whether the single cell has an aneuploidy with respect to the first chromosome, based on a difference between the first parameter and a preset control parameter. In the sequencing result based on the whole genome sequencing of the single cell, the number of sequencing data for a certain chromosome positively correlates to a content of the chromosome in the genome, thus, analyzing the number of sequencing data deriving from the certain chromosome and the total number of the whole genome sequencing in the sequencing result may effectively determine whether the single cell has an aneuploidy with respect to the certain chromosome.

According to some embodiments of the present disclosure, the above-mentioned method of determining the chromosome aneuploidy of the single cell may also have following additional technical features:

According to an embodiment of the present disclosure, the method of determining the chromosome aneuploidy of the single cell may further comprise a step of isolating the single cell from a biological sample. Thus, the method of determining the chromosome aneuploidy of the single cell according to embodiments of the present disclosure may obtain information regarding whether the biological sample having a chromosome aberration directly taken the biological sample as a raw material, which may reflect a healthy status of a living body.

According to an embodiment of the present disclosure, the biological sample is at least one selected from a group consisting of blood, urine, saliva, tissue, germ cell, blastomere, and embryo. Thus, the method of determining the chromosome aneuploidy of the single cell according to embodiments of the present disclosure may conveniently obtain these samples from the living body, and may select different sample specifically in accordance with certain diseases, so as to adopt a certain analyzing result for the certain diseases.

According to an embodiment of the present disclosure, isolating the single cell from the biological sample is performed using at least one selected from a group consisting of dilution, mouth-controlled pipette isolation, micromanipulation, flow cytometry isolation, and microfluidic. According to one specific example of the present disclosure, preferably the micromanipulation is micro-dissection. Thus, the method of determining the chromosome aneuploidy of the single cell according to embodiments of the present disclosure may effectively and conveniently obtain the single cell deriving from the biological sample, to perform the subsequent operations, which may improve the efficiency of determining the chromosome aneuploidy of the single cell.

According to an embodiment of the present disclosure, sequencing the whole genome of the single cell may further comprise: amplifying the whole genome of the single cell to obtain an amplified whole genome; constructing a whole genome sequencing-library using the amplified whole genome; and sequencing the whole genome sequencing-library to obtain a plurality of sequencing data, wherein the plurality of sequencing data constitute the first sequencing result. According to one specific example of the present disclosure, sequencing the whole genome of the single cell may further comprise a step of lysing the single cell to release the whole genome of the single cell. Thus, the method of determining the chromosome aneuploidy of the single cell according to embodiments of the present disclosure may effectively obtain information of the whole genome deriving from the single cell, so as to further improve the efficiency of determining the chromosome aneuploidy of the single cell.

According to an embodiment of the present disclosure, lysing the single cell to release the whole genome of the single cell is performed using an alkaline lysis buffer. Thus, the method of determining the chromosome aneuploidy of the single cell according to embodiments of the present disclosure may effectively lyse the single cell, so as to further improve the efficiency of determining the chromosome aneuploidy of the single cell.

According to an embodiment of the present disclosure, amplifying the whole genome is performed using a PCR-based whole genome amplification method. According to one specific example of the present disclosure, the PCR-based whole genome amplification method is OmniPlex WGA. Thus, the method of determining the chromosome aneuploidy of the single cell according to embodiments of the present disclosure may effectively amplify the whole genome, so as to further improve the efficiency of determining the chromosome aneuploidy of the single cell.

According to an embodiment of the present disclosure, sequencing the whole genome sequencing-library is performed using at least one selected from a group consisting of Hiseq2000, SOLiD, Roche 454, and single-molecule sequencing apparatus. Thus, the characteristics of high-throughput and deep sequencing of these sequencing apparatuses may be utilized, to further improve the efficiency of determining the chromosome aneuploidy of the single cell.

According to an embodiment of the present disclosure, the plurality of sequencing data has an average length of about 50 bp. Thus, the method of determining the chromosome aneuploidy of the single cell according to embodiments of the present disclosure may conveniently analyze sequencing data and improve the analyzing efficiency, so as to further improve the efficiency of determining the chromosome aneuploidy of the single cell.

According to an embodiment of the present disclosure, the method of determining the chromosome aneuploidy of the single cell according to embodiments of the present disclosure may further comprise a step of aligning the first sequencing result to known genomic sequence information, to obtain all sequencing data which can be aligned to the known genomic sequence and sequencing data deriving from the first chromosome. Thus, the method of determining the chromosome aneuploidy of the single cell according to embodiments of the present disclosure may effectively determine sequencing data deriving from the certain chromosome, so as to further improve the efficiency of determining the chromosome aneuploidy of the single cell.

According to an embodiment of the present disclosure, the first chromosome is at least one selected from human chromosome 21, chromosome 18, chromosome 13, chromosome X and chromosome Y. Thus, the chromosomal diseases common in human may be effectively determined, for example, a fetal genetic disease may be predetermined.

According to an embodiment of the present disclosure, the first parameter is a ratio M/L of the value M to the value L. Thus, the method of determining the chromosome aneuploidy of the single cell according to embodiments of the present disclosure may conveniently analyze the sequencing result, so as to further improve the efficiency of determining the chromosome aneuploidy of the single cell.

According to an embodiment of the present disclosure, the preset control parameter is obtained by following steps of: sequencing a whole genome of a control single cell to obtain a second sequencing result, in which the whole genome of the control single cell derives from a sample without the chromosome aneuploidy; counting the total number of sequencing data which can be aligned to a reference genome in sequencing data of the second sequencing result, to obtain a value L′; counting the number of sequencing data which can be aligned to the first chromosome of the reference genome in the second sequencing result, to obtain a value M′; and determining a ratio M′/L′ of the value M′ to the value L′, to obtain the preset control parameter. Thus, the method of determining the chromosome aneuploidy of the single cell according to embodiments of the present disclosure may conveniently determine the control parameter, so as to further improve the efficiency of determining the chromosome aneuploidy of the single cell.

According to an embodiment of the present disclosure, in the case of a ratio of the first parameter to the preset control parameter exceeding a first threshold, the number of the first chromosome of the single cell is determined to be 3; in the case of the ratio of the first parameter to the preset control parameter falling below a second threshold, the number of the first chromosome of the single cell is determined to be 1; and in the case of the ratio of the first parameter to the preset control parameter being between the first threshold and the second threshold, the number of the first chromosome of the single cell is determined to be 2. Thus, the method of determining the chromosome aneuploidy of the single cell according to embodiments of the present disclosure may rapidly predetermine that whether the number of the certain chromosome has an abnormality.

According to an embodiment of the present disclosure, the method of determining the chromosome aneuploidy of the single cell according to embodiments of the present disclosure may further comprise a step of subjecting the ratio of the first parameter to the preset control parameter to a Student's t-test, to obtain a Student's t-test value of the first chromosome. Thus, the accuracy and the precision of analyzing the sequencing result may further improved.

According to another aspect of the present disclosure, there is provided a system for determining a chromosome aneuploidy of a single cell. According to embodiments of the present disclosure, the system for determining the chromosome aneuploidy of the single cell may comprise: a whole genome sequencing apparatus, for sequencing a whole genome of the single cell to obtain a first sequencing result; a sequencing result analyzing apparatus, connected to the whole genome sequencing apparatus, for receiving the first sequencing result from the whole genome sequencing apparatus to perform following steps: counting the total number of sequencing data which can be aligned to a reference genome in sequencing data of the first sequencing result, to obtain a value L; counting the number of sequencing data which can be aligned to a first chromosome in the reference genome in the first sequencing result, to obtain a value M; determining a first parameter based on the value L and the value M; determining whether the single cell has an aneuploidy with respect to the first chromosome, based on a difference between the first parameter and a preset control parameter. Thus, utilizing the system for determining the chromosome aneuploidy of the single cell may effectively implement the method of determining the chromosome aneuploidy of the single cell according to embodiments of the present disclosure, so as to effectively determine the chromosome aneuploidy of the single cell.

According to some embodiments of the present disclosure, the system for determining the chromosome aneuploidy of the single cell may further have following additional technical features:

According to an embodiment of the present disclosure, the system for determining the chromosome aneuploidy of the single cell may further comprise a whole genome sequencing-library constructing apparatus, connected to the whole genome sequencing apparatus, to provide the whole genome sequencing-library for sequencing to the whole genome sequencing apparatus, wherein the whole genome sequencing-library constructing apparatus may further comprise: a single cell isolating unit, for isolating the single cell from a biological sample; a single cell lysing unit, for receiving an isolated single cell and lysing the single cell, to release the whole genome of the single cell; a whole genome amplifying unit, connected to the single cell lysing unit, for receiving the whole genome of the single cell and amplifying the whole genome of the single cell; and a sequencing-library constructing unit, for receiving an amplified whole genome, and constructing the whole genome sequencing-library using the amplified whole genome. Thus, whole genome information of the single cell may be effectively obtained, which may further improve the efficiency of determining the chromosome aneuploidy of the single cell.

According to an embodiment of the present disclosure, the single cell isolating unit comprises at least one apparatus suitable for performing following operations selected from a group consisting of dilution, mouth-controlled pipette isolation, micromanipulation, flow cytometry isolation, and microfluidic. According to one specific example of the present disclosure, the micromanipulation preferably is micro-dissection. Thus, the single cell deriving from the biological sample may be effectively and conveniently obtained, which may improve the efficiency of determining the chromosome aneuploidy of the single cell.

According to an embodiment of the present disclosure, the single cell lysing unit comprises an apparatus suitable for lysing the single cell using an alkaline lysis buffer. Thus, the single cell lysing unit may effectively lyse and release the whole genome of the single cell, so as to improve the efficiency of determining the chromosome aneuploidy of the single cell.

According to an embodiment of the present disclosure, the whole genome amplifying unit may comprise an apparatus suitable for amplifying the whole genome using a PCR-based whole genome amplification method. According to one specific example of the present disclosure, the PCR-based whole genome amplification method is OmniPlex WGA. Thus, the whole genome amplifying unit may effectively amplify the whole genome, so as to further improve the efficiency of determining the chromosome aneuploidy of the single cell.

According to an embodiment of the present disclosure, the whole genome sequencing apparatus comprises at least one selected from a group consisting of Hiseq2000, SOLiD, Roche 454, and single-molecule sequencing apparatus. Thus, the characteristics of high-throughput and deep sequencing of these sequencing apparatuses may be utilized, to further improve the efficiency of determining the chromosome aneuploidy of the single cell.

According to an embodiment of the present disclosure, the sequencing result analyzing apparatus may further comprise a sequence aligning unit, for aligning the first sequencing result to known genomic sequence information, to obtain all sequencing data which can be aligned to the reference genome, and to obtain sequencing data deriving from the first chromosome. Thus, the system of determining the chromosome aneuploidy of the single cell according to embodiments of the present disclosure may effectively determine the sequencing data deriving from the certain chromosome, so as to further improve the efficiency of determining the chromosome aneuploidy of the single cell.

According to an embodiment of the present disclosure, the sequencing result analyzing apparatus may further comprise a Student's t-test unit, for subjecting a ratio of the first parameter to the preset control parameter to a Student's t-test, to obtain a Student's t-test value of the first chromosome. Thus, the accuracy and the precision of analyzing the sequencing result may be further improved.

Additional aspects and advantages of embodiments of present disclosure will be given in part in the following descriptions, become apparent in part from the following descriptions, or be learned from the practice of the embodiments of the present disclosure.

### BRIEF DESCRIPTION OF THE DRAWINGS

These and other aspects and advantages of embodiments of the present disclosure will become apparent and more readily appreciated from the following descriptions made with reference to the accompanying drawings, in which:

FIG. 1 shows a flow chart of a method of determining a chromosome aneuploidy of a single cell according to an embodiment of the present disclosure.

FIG. 2 shows a schematic diagram of a system for determining a chromosome aneuploidy of a single cell according to an embodiment of the present disclosure.

FIG. 3 shows a schematic diagram of a system for determining a chromosome aneuploidy of a single cell according to another embodiment of the present disclosure.

FIG. 4 shows a schematic diagram of an apparatus for constructing a whole genome sequencing-library according to an embodiment of the present disclosure.

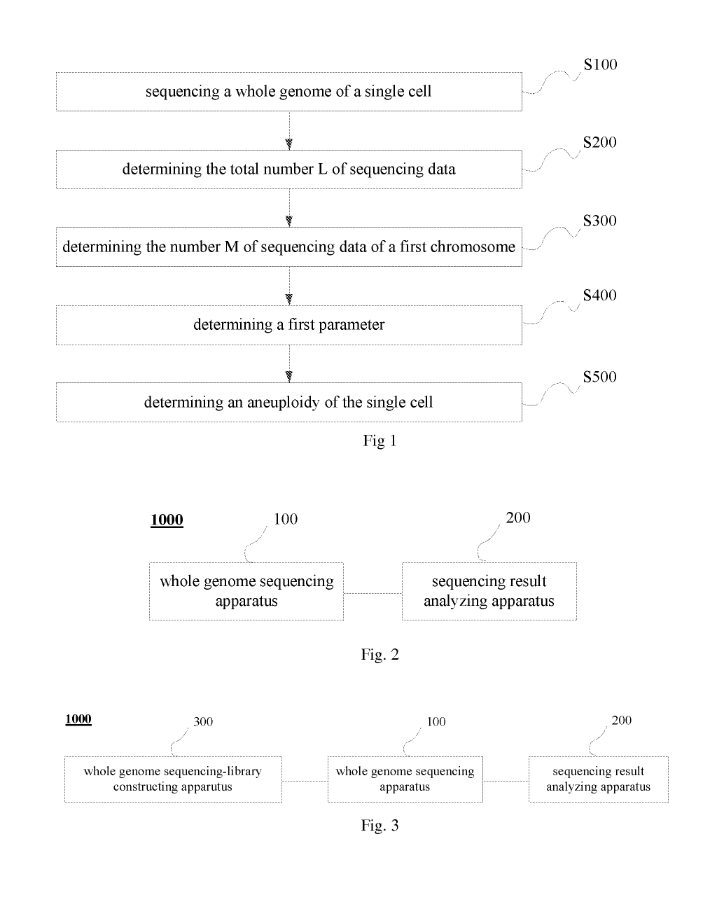

### DETAILED DESCRIPTION

Reference will be made in detail to embodiments of the present disclosure, the examples of the embodiments will be shown in Figures, in which the same or similar elements and the elements having same or similar functions are denoted by like reference numerals throughout the descriptions. The embodiments described herein with reference to drawings are explanatory, illustrative, and used to generally understand the present disclosure. The embodiments shall not be construed to limit the present disclosure.

In addition, terms such as “first” and “second” are used herein for purposes of description and are not intended to indicate or imply relative importance or significance. Thus, features restricted with “first”, “second” may explicitly or implicitly comprise one or more of the features. Furthermore, in the description of the present disclosure, unless otherwise stated, the term “a plurality of” refers to two or more. A term “aneuploidy” used herein is a relative term with euploidy of a chromosome, which refers to one or more chromosome missing or extra adding in the genome. In general, there are two chromosomes in each type in normal cells, however, a gamete having an abnormal number of chromosomes formed by non-segregating or segregating a pair of homologous chromosomes in advance during meiosis phase, a variety of aneuploidy cells will generate when the above-mentioned gamete combines each other or combines with a normal gamete. In addition, the aneuploidy cells will also generate during somatic cell division, such as a tumor cell having a high aberration rate, etc.

One aspect of the present disclosure provides a method which may effectively determine a chromosome aneuploidy of a single cell. The method of determining the chromosome aneuploidy of the single cell according to embodiments of the present disclosure may comprise following steps:

S100: sequencing a whole genome of the single cell to obtain a first sequencing result

According to embodiments of the present disclosure, a source of the single cell is not subjected to any special restriction. According to some embodiments of the present disclosure, the single cell may be isolated from a biological sample. Furthermore, according to an embodiment of the present disclosure, the method of determining the chromosome aneuploidy of the single cell may further comprise a step of isolating the single cell from a biological sample. Thus, the method of determining the chromosome aneuploidy of the single cell according to embodiments of the present disclosure may obtain information regarding whether the biological sample having a chromosome aberration directly taken the biological sample as a raw material, which may reflect a healthy status of a living body. According to embodiments of the present disclosure, the used biological sample is not subjected to any special restriction. According to some specific examples of the present disclosure, the biological sample may be at least one selected from a group consisting of blood, urine, saliva, tissue, germ cell, blastomere, and embryo. It would be appreciated by those skilled in the art that, for different diseases, different biological samples may be used to perform the analysis. Thus, these samples may be conveniently obtained from a living body, and different samples may be used specifically for certain diseases, so as to select a certain analyzing method for these diseases. For example, for those subjects to be tested which may suffer a certain cancer, a sample may be collected from a tissue of a lesion or surroundings thereof, from which a single cell is isolated for analysis, thus, whether the cancer happens in the tissue may be precisely aware as early as possible. According to embodiments of the present disclosure, the used method and apparatus for isolating the single cell from the biological sample are not subjected to any special restriction. According to some specific examples of the present disclosure, the single cell is isolated from the biological sample using at least one selected from a group consisting of dilution, mouth-controlled pipette isolation, micromanipulation (micro-dissection is preferred), flow cytometry isolation, and microfluidic. Thus, the method of determining the chromosome aneuploidy of the single cell according to embodiments of the present disclosure may effectively and conveniently obtain the single cell deriving from the biological sample, to perform the subsequent operations, which may improve the efficiency of determining the chromosome aneuploidy of the single cell.

In addition, according to embodiments of the present disclosure, the used method of sequencing the whole genome of the single cell is not subjected to any special restrictions. According to an embodiment of the present disclosure, sequencing the whole genome of the single cell may further comprise: firstly, amplifying the whole genome of the single cell to obtain an amplified whole genome; then, constructing a whole genome sequencing-library using the amplified whole genome; and finally sequencing the whole genome sequencing-library to obtain a plurality of sequencing data, in which the plurality of sequencing data constitute the first sequencing result. Thus, the method of determining the chromosome aneuploidy of the single cell according to embodiments of the present disclosure may effectively obtain information of the whole genome deriving from the single cell, so as to further improve the efficiency of determining the chromosome aneuploidy of the single cell. Those skilled in the art may select different methods of constructing the whole genome sequencing-library in accordance with the specific solution of the genome sequencing technique used, details of constructing the whole genome sequencing-library may refer to a specification provided by sequencing-instrument manufacturer, such as Illumina company, for example Multiplexing Sample Preparation Guide (Part#1005361; February 2010) or Paired-End SamplePrep Guide (Part#1005063; February 2010) is referred, which are both incorporated herein by reference.

Optionally, according to embodiments of the present disclosure, a step of lysing the single cell to release the whole genome of the single cell may be further comprised. According to some embodiments of the present disclosure, the used method for lysing the single cell to release the whole genome is not subjected to any special restrictions, as long as the used method may sufficiently lyse the single cell. According to specific examples of the present disclosure, the single cell is lysed to release the whole genome of the single cell using an alkaline lysis buffer. The inventors find out that, the single cell may be effectively lysed to release the whole genome, and the released whole genome may provide a high accuracy during the process of sequencing, so as to further improve the efficiency of determining the chromosome aneuploidy of the single cell. According to embodiment of the present disclosure, the used method of amplifying the whole genome of the single cell is not subjected to any special restrictions, the whole genome is amplified using a PCR-based method, for example, PEP-PCR, DOP-PCR and OmniPlex WGA may be used, or the whole genome is amplified using a method other than the PCR-based method such as MDA (multiple displacement amplification). According to specific examples of the present disclosure, the PCR-based method is preferably used, for example, the PCR-based method is OmniPlex WGA. The commercial kit optional used may comprise, but not limited to GenomePlex from Sigma Aldrich, PicoPlex from Rubicon Genomics, and illustra GenomiPhi from GE Healthcare, etc. Thus, according to specific embodiments of the present disclosure, before the step of constructing the sequencing-library, the whole genome of the single cell may be amplified using OmniPlex WGA. Thus, the whole genome may be effectively amplified, so as to further improve the efficiency of determining the chromosome aneuploidy of the single cell. According to embodiments of the present disclosure, the whole genome sequencing-library is sequenced using at least one selected from a group consisting of Hiseq2000, SOLiD, Roche 454, and single-molecule sequencing apparatus. Thus, the characteristics of high-throughput and deep sequencing of these sequencing apparatuses may be utilized, to further improve the efficiency of determining the chromosome aneuploidy of the single cell. Obviously, it would be appreciated by those skilled in the art that, other sequencing methods and apparatuses may be used in sequencing the whole genome, for example a Third-Generation sequencing technique, and more advanced sequencing technique which may be developed later. According to embodiments of the present disclosure, the plurality of sequencing data has an average length of about 50 bp. The inventors surprisingly find out that, sequencing data having an average length of about 50 bp may greatly facilitate analyzing sequencing data, to improve the efficiency of analysis, at the same time the cost of analysis may be also significantly reduced. The efficiency of determining the chromosome aneuploidy of the single cell may be further improved, and the cost of determining the chromosome aneuploidy of the single cell may also be reduced. The term “average length” used herein refers to an average value among the length value of each sequencing data.

S200: counting the total number of sequencing data which can be aligned to a reference genome in the first sequencing result, to obtain a value L

After the step of sequencing the whole genome of the single cell is completed, the obtained sequencing result comprises a plurality of sequencing data. The term “sequencing data which can be aligned to a reference genome” refers to sequencing data which may be aligned to the reference genome, by means of aligning all sequencing data of the sequencing result to a known reference genome sequence (for example human genome Hgl 9). It would be appreciated by those skilled in the art that any known methods may be used to counting the total number of these sequencing data. For example, software provided by sequencing-instrument manufacturer may be used for analysis.

S300: counting the number of sequencing data which can be aligned to a first chromosome in the reference genome in the first sequencing result, to obtain a value M

The term “first chromosome” used herein should be broadly understood, which may refer to any chromosome desired to be investigated, the number thereof is not limited to one chromosome, and all chromosomes may be subjected to analyzing even at the same time. According to embodiments of the present disclosure, the first chromosome may be any chromosome in human chromosome, which may be any chromosome selected from human chromosome 1 to chromosome 23. According to embodiments of the present disclosure, preferably the first chromosome is at least one selected from human chromosome 21, chromosome 18, chromosome 13, chromosome X and chromosome Y. Thus, the chromosomal diseases common in human may be effectively determined, for example, a fetal genetic disease may be predetermined. Therefore, the method of determining the chromosome aneuploidy of the single cell according to embodiments of the present disclosure may be very effectively applied to pre-implantation genetic screening (PGS), pre-implantation genetic diagnosis (PGD), prenatal testing of fetal nucleated red blood cells and etc. in the field of in vitro fertilization (IVF), or may be applied to prenatal testing of the fetal single cell extracted from amniotic fluid of pregnant women. Thus, whether the fetal chromosome has an abnormality may be rapidly predetermined by means of simply extracting the single cell, to avoid severe genetic disease suffered by fetus. The term “can be aligned to a first chromosome in the reference genome” refers to a sequencing result that sequencing data deriving from the first chromosome can be determined, by means of aligning these sequencing data to a known sequence of the first chromosome in the reference genome, in which the sequencing data can be aligned to the known sequence of the first chromosome.

According to embodiments of the present disclosure, the used method of screening sequencing data deriving from a certain chromosome in the first sequencing result is not subjected to any special restrictions. According to a specific example of the present disclosure, sequencing data deriving from the first chromosome may be screened out, by means of aligning the first sequencing result to known genomic sequence information. Thus, according to an embodiment of the present disclosure, the method of determining the chromosome aneuploidy of the single cell may further comprise a step of aligning the first sequencing result to known genomic sequence information using conventional software, to screen out the sequencing data deriving from the first chromosome. Thus, the sequencing data deriving from a certain chromosome may be effectively determined, so as to further improve the efficiency of determining the chromosome aneuploidy of the single cell.

S400: determining a first parameter based on the value L and the value M

According to embodiments of the present disclosure, the value L and the value M may be subjected to any conventional mathematical calculation and statistical analysis, and obtained result may be subjected to a preset control parameter, to determine whether the chromosome represented by the value M has aneuploidy information. Relative data volume regarding data volume of a certain chromosome to total sequencing data volume, namely a ratio between the data volumes of the certain chromosome and the total data volume, which may be calculated within a range of an intact chromosome, or may be calculated by means of artificially dividing the intact chromosome into windows, in which a size of the window may be fixed or not fixed. A type of data volume may comprise but not limited to the number of reads, the number of bases, depth, average depth, coverage, and etc. According to an embodiment of the present disclosure, the first parameter is a ratio M/L of the value M to the value L. The inventors find out that the obtained value by means of simple mathematical operation may reflect relevant information regarding the content of the certain chromosome in the whole genome. Thus, the sequencing result may be conveniently analyzed, which may improve the efficiency of determining the chromosome aneuploidy of the single cell.

S500: determining whether the single cell has an aneuploidy with respect to the first chromosome, based on a difference between the first parameter and a preset control parameter

According to embodiments of the present disclosure, whether the single cell has an aneuploidy with respect to the first chromosome may be determined based on a difference between the first parameter and a preset control parameter, by means of aligning the above-determined first parameter to the preset control parameter. In the sequencing result based on the whole genome sequencing of the single cell, the number of sequencing data for a certain chromosome positively correlates to a content of the chromosome in the genome, thus, analyzing the number of sequencing data deriving from the certain chromosome and the total number of the whole genome sequencing in the sequencing result may effectively determine whether the single cell has an aneuploidy with respect to the certain chromosome. The term “control parameter” used herein refers to relevant data regarding a certain chromosome obtained by subjecting a nucleic acid sample with a genome known to be normal to repeating the protocol and analysis conducted to a single cell of a biological sample. It would be appreciated by those skilled in the art that a relevant parameter of a certain chromosome and a relevant parameter of a chromosome from a normal nucleic acid sample may be obtained using a same condition for sequencing and a same mathematics method, respectively. Here, the relevant parameter of the chromosome from the normal nucleic acid sample may be taken as a control reference. In addition, the term “preset” used herein should be broadly understood, which may be determined by an experiment in advance, or may be obtained from a parallel experiment when performing analysis with the biological sample. The term “parallel experiment” used herein should be broadly understood, which may refer to subjecting an unknown sample and a known sample to sequencing and analysis simultaneously, or to sequencing and analysis in succession under the same conditions. According to embodiments of the present disclosure, when the ratio M/L of the value M to the value L is taken as the first parameter, the control parameter value may be determined using following methods: firstly, sequencing a whole genome of a control single cell to obtain a second sequencing result, in which the whole genome of the control single cell derives from a sample without the chromosome aneuploidy; secondly, counting the total number of sequencing data which can be aligned to a reference genome in sequencing data of the second sequencing result, to obtain a value L′; thirdly, counting the number of sequencing data which can be aligned to the first chromosome of the reference genome in the second sequencing result, to obtain a value M′; and finally determining a ratio M′/L′ of the value M′ to the value L′, the obtained ratio M′/L′ may be taken as the preset control parameter. Thus, the control parameter may conveniently determine, and the efficiency of determining the chromosome aneuploidy of the single cell may be improved.

To determine the difference between the first parameter and the preset control parameter, those skilled in the art may use any known mathematical operation to perform. According to embodiments of the present disclosure, the inventors find out that the ratio of the first parameter and the control parameter may be firstly obtained, then the information regarding the aneuploidy of the certain chromosome is obtained by comparing the obtained ratio with a first threshold and a second threshold which are both predetermined The terms “first threshold” and “second threshold” used herein respectively reflects a value of an extra added chromosome or a missed chromosome, those skilled in the art may determine these values by subjecting a sample having known genomic sequence information to relevant serial experiments, for example, by subjecting the sample extracted from a fetus suffering Down Syndrome to the above experiment, to obtain a threshold regarding human chromosome 21 under a condition of having an extra added chromosome, namely the first threshold, other pathological samples may also be used to determine a threshold under a condition of missing one chromosome, namely the second threshold. According to an embodiment of the present disclosure, the value of the first threshold may be about 1.25-1.75, for example may be about 1.5, the second threshold may be about 0.25-0.75, for example may be about 0.5. Thus, according to an embodiment of the present disclosure, in the case of a ratio of the first parameter to the preset control parameter exceeding a first threshold, the number of the first chromosome of the single cell is determined to be 3, i.e. having an extra added chromosome; in the case of the ratio of the first parameter to the preset control parameter falling below a second threshold, the number of the first chromosome of the single cell is determined to be 1; and in the case of the ratio of the first parameter to the preset control parameter being between the first threshold and the second threshold, the number of the first chromosome of the single cell is determined to be 2. Thus, whether the number of the certain chromosome has an abnormality may be rapidly determined by setting the first threshold and the second threshold. In addition, according to embodiments of the present disclosure, the ratio of the first parameter to the preset control parameter may be subjected to mathematical statistical tests for example, a Student's t-test, to improve the accuracy and the precision of analyzing the sequencing result. It would be appreciated by those skilled in the art that, after performing the relevant mathematical statistical tests, a different first threshold and a different second threshold may be set accordingly, to perform an analysis similar with the above.

According to another aspect of the present disclosure, there is provided a system 1000 for determining a chromosome aneuploidy of a single cell. Referring to FIG. 2 to FIG. 4, according to embodiments of the present disclosure, the system 1000 for determining the chromosome aneuploidy of the single cell may comprise: a whole genome sequencing apparatus 100 and a sequencing result analyzing apparatus 200. According to embodiments of the present disclosure, the whole genome sequencing apparatus 100 is used for sequencing a whole genome of the single cell to obtain a first sequencing result; and the sequencing result analyzing apparatus 200 receives the first sequencing result from the whole genome sequencing apparatus 100. The sequencing result analyzing apparatus 200 may perform following operations: firstly, counting the total number of sequencing data which can be aligned to a reference genome in sequencing data of the first sequencing result, to obtain a value L; secondly, counting the number of sequencing data which can be aligned to a first chromosome in the reference genome in the first sequencing result, to obtain a value M; determining a first parameter based on the value L and the value M; thirdly, determining whether the single cell has an aneuploidy with respect to the first chromosome, based on a difference between the first parameter and a preset control parameter. Thus, utilizing the system 1000 for determining the chromosome aneuploidy of the single cell may effectively implement the method of determining the chromosome aneuploidy of the single cell according to embodiments of the present disclosure, so the chromosome aneuploidy of the single cell may be effectively determined

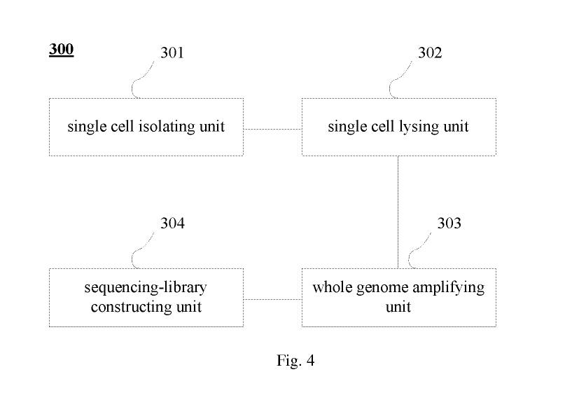

Referring to FIG. 3, according to an embodiment of the present disclosure, the system 1000 for determining the chromosome aneuploidy of the single cell may further comprise a whole genome sequencing-library constructing apparatus 300. According to embodiments of the present disclosure, the whole genome sequencing-library constructing apparatus 300 provides the whole genome sequencing-library for sequencing to the whole genome sequencing apparatus 100. Referring to FIG. 4, the whole genome sequencing-library constructing apparatus 300 may further comprise: a single cell isolating unit 301, a single cell lysing unit 302, a whole genome amplifying unit 303, and a sequencing-library constructing unit 304. According to embodiments of the present disclosure, the single cell isolating unit 301 is used for isolating the single cell from a biological sample; the single cell lysing unit 302 is used for receiving an isolated single cell and lysing the single cell, to release the whole genome of the single cell; the whole genome amplifying unit 303, connected to the single cell lysing unit 302, is used for receiving the whole genome of the single cell and amplifying the whole genome of the single cell; and the sequencing-library constructing unit 304, connected to the whole genome amplifying unit 303, is used for receiving an amplified whole genome, and constructing the whole genome sequencing-library using the amplified whole genome. Thus, the whole genome information of the single cell may be effectively obtained, which may further improve the efficiency of determining the chromosome aneuploidy of the single cell. The term “connect” used herein should be broadly understood, which may be a direct connection, or an indirect connection, or even may be by means of one container or apparatus, as long as the connection of the above functions can be achieved, for example, the single cell lysing unit 302 and the whole genome amplifying unit 303 may be conducted in one apparatus, i.e. after the single cell is lysed to release the whole genome, the released whole genome may be subjected to the whole genome amplification in the identical apparatus or container which is used for the lysing step, without being transferred to other apparatus or container, just by means of converting a condition in the apparatus (comprising a reaction condition and a reaction system) to a condition suitable for a reaction of the whole genome amplification. That will achieve a functional connection between the single cell lysing unit 302 and the whole genome amplifying unit 303, which is regarded as being covered by the term “connect”.

According to an embodiment of the present disclosure, the single cell isolating unit 301 comprises at least one apparatus suitable for performing following operations selected from a group consisting of dilution, mouth-controlled pipette isolation, micromanipulation, flow cytometry isolation, and microfluidic. According to one specific example of the present disclosure, the micromanipulation preferably is micro-dissection. Thus, the single cell deriving from the biological sample may be effectively and conveniently obtained, which may improve the efficiency of determining the chromosome aneuploidy of the single cell. Those skilled in the art may select different methods and apparatuses for constructing the whole genome sequencing-library in accordance with the specific solution of the genome sequencing technique used. Details of constructing the whole genome sequencing-library may refer to a specification provided by sequencing-instrument manufacturer. According to some embodiment of the present disclosure, the used method for lysing the single cell to release the whole genome is not subjected to any special restrictions, as long as the used method may sufficiently lyse the single cell. According to specific examples of the present disclosure, the single cell is lysed to release the whole genome of the single cell using an alkaline lysis buffer. The inventors find out that, the whole genome of the single cell may effectively be release, and the efficiency of subjecting the obtained whole genome to sequencing may be improved, so as to further improve the efficiency of determining the chromosome aneuploidy of the single cell. Thus, according to an embodiment of the present disclosure, the single cell lysing unit 302 comprises an apparatus suitable for performing cell lysis to obtain the whole genome (not shown in Figure). Thus, the whole genome of the single cell may be effectively obtained, so as to further improve the efficiency of determining the chromosome aneuploidy of the single cell. According to an embodiment of the present disclosure, the whole genome amplifying unit 303 comprises an apparatus suitable for performing the whole genome amplification using OmniPlex WGA. Thus, the whole genome may effectively be amplified, so as to further improve the efficiency of determining the chromosome aneuploidy of the single cell.

According to an embodiment of the present disclosure, the whole genome sequencing apparatus 100 comprises at least one selected from a group consisting of Hiseq2000, SOLiD, Roche 454, and single-molecule sequencing apparatus. Thus, the characteristics of high-throughput and deep sequencing of these sequencing apparatuses may be utilized, to further improve the efficiency of determining the chromosome aneuploidy of the single cell. Obviously, it would be appreciated by those skilled in the art that, other sequencing methods and apparatuses may be used in sequencing the whole genome, for example a Third-Generation sequencing technique, and more advanced sequencing technique which may be developed later. According to embodiments of the present disclosure, the length of sequencing data obtained by the whole genome sequencing is not subjected to any special restriction. According to a specific example of the present disclosure, sequencing data has an average length of about 50 bp.

According to an embodiment of the present disclosure, the sequencing result analyzing apparatus 200 further comprises a sequence aligning unit (not shown in Figure), which is used for aligning the first sequencing result to known genomic sequence information, to obtain all sequencing data which can be aligned to the reference genome, and to obtain sequencing data deriving from the first chromosome. Thus, the sequencing data deriving from the certain chromosome may be effectively determined. The term “first chromosome” used herein should be broadly understood, which may refer to any chromosome desired to be investigated, the number thereof is not limited to one chromosome, and all chromosomes may be subjected to analyzing even at the same time. According to embodiments of the present disclosure, the first chromosome may be any chromosome in the human chromosomes, for example, the first chromosome is at least one selected from human chromosome 21, chromosome 18, chromosome 13, chromosome X and chromosome Y. Thus, the chromosomal diseases common in human may be effectively determined, for example, a fetal genetic disease may be predetermined. Therefore, the method of determining the chromosome aneuploidy of the single cell according to embodiments of the present disclosure may be very effectively applied to pre-implantation genetic screening (PGS), pre-implantation genetic diagnosis (PGD), prenatal testing of fetal nucleated red blood cells and etc. in the field of in vitro fertilization (IVF), or may be applied to prenatal testing of the fetal single cell extracted from amniotic fluid of pregnant women. Thus, whether the fetal chromosome has an abnormality may be rapidly predetermined by means of simply extracting the single cell, to avoid severe genetic disease suffered by fetus.

As described above in detail, the chromosome aneuploidy is subjected to analysis based on based on the value L and the value M, so a detailed description thereof will be omitted here. It should be noted that according to an embodiment of the present disclosure, the sequencing result analyzing apparatus 200 may further comprise a Student's t-test unit, for subjecting a ratio of the first parameter to the preset control parameter to a Student's t-test, or for respectively subjecting the first parameter and the preset control parameter to a Student's t-test, to obtain a Student's t-test value of the first chromosome. Thus, the accuracy and the precision of analyzing the sequencing result may further improved.

Reference will be made in detail to examples of the present disclosure. It should be noted that the following examples are explanatory, and cannot be construed to limit the scope of the present disclosure.

Experimental Material

A single cell collected from a normal male blood (abbreviated as YH blood sample) was taken as a single cell from the normal control blood. A single cell of a sample to be tested was from a single cell of a female blood (abbreviated as T21 blood sample) who suffered Down syndrome (having three human chromosomes 21). Unless specifically stated, other used experimental materials were a reagent which was formulated according to a conventional method in the art, or a reagent which was commercially available.

Experiment Protocol

1. Single Cell Separation.

The YH blood sample and the T21 blood sample were centrifuged to isolate a leukocyte layer. After rinsed by PBS, the leukocyte was suspended in droplets of PBS, then the single leukocyte was separated using a mouth-controlled pipette and placed in a 1˜2 μL of alkaline lysis buffer, and then was frozen and stored at −20° C. for 30 min or more. 3 single cells were isolated respectively from the YH blood sample and the T21 blood sample (respectively labeled with YHSigm-1, YHSigm-2, YHSigm-3, T21Sigm-1, T21Sigm-2, and T21Sigm-3).

2. Single Cell Lysis and Whole Genome Amplification

The single cell placed in the alkaline lysis buffer was subjected to 65° C. for 5 to 15 min, to lyse the single cell. Then the lysed single cell was subjected to whole genome amplification using GenomePlex WGA kit of Sigma Aldrich, specific operations may refer to GenomePlex Single Cell Whole Genome Amplification Kit (WGA4)-Technical Bulletin (PHC 09/10-1), which was incorporated here by reference. In short, firstly a single cell whole genome DNA was randomly broken, for constructing an OmniPlex library having a ligation region of a universal primer at both sides, then the obtained OmniPlex library was subjected to a PCR amplification with limited cycle, namely the single cell whole genome amplification was completed.

3. Whole Genome Sequencing-Library Construction

According to Paired-End SamplePrep Guide (Part#1005063; February 2010), which was incorporated herein by reference, the whole genome sequencing-library of an inserted fragment having a length of about 150 bp was constructed using Illumina Paired-End DNA Sample Prep Kit.

4. High-Throughput Sequencing

The high-throughput sequencing was performed using Illumina Hiseq2000 sequencing system. The constructed whole genome sequencing-library was subjected to cluster generation using cBot, then the obtained clustering sequencing-library was subjected to Hiseq2000 sequencer, the sequencing length was 50 bp.

5. Data Alignment to a Reference Genome

The reads data obtained by sequencing was aligned to a reference genome using SOAP software, HG 18 was taken as a human reference genomic sequence, an obtained aligning result was calculated on a condition of allowing a mismatching of 2 bases. Table 1 showed a calculation result of the aligning result, each single cell obtained 11.7-14.6 M of the reads number, an aligning ratio was with a range of 68% and 76%, an aligning ratio which can be uniquely aligned was to a range of 75% and 80%. Comparing with the whole genome DNA sequencing, the aligning ratio of the single cell WGA data was low, which resulted from a combination deviation of a degenerated sequence in a primer during the PCR amplification using GenomePlex WGA. Since the deviation part could not be aligned to the reference sequence, those data which could be aligned to the reference sequencing would not be affected.

TABLE 1
Calculation of result obtained by data alignment

| | | | | | Alignment |
|---|---|---|---|---|---|
| | | | | Reads | ratio |
| | | | | which | which |
| | | | | can be | can be |
| | Total | Reads can | Alignment | uniquely | uniquely |
| Sample | Reads | be aligned | ratio | aligned | alighed |
| T21Sigm-1 | 13407381 | 9217701 | 68.70% | 7341230 | 80% |
| T21Sigm-2 | 14641324 | 11200230 | 76.50% | 9023423 | 80% |
| T21Sigm-3 | 11940348 | 8320190 | 69.70% | 6650907 | 80% |
| YHSigm-1 | 11747486 | 8607725 | 73.30% | 6552207 | 76% |
| YHSigm-2 | 14319331 | 10226897 | 71.40% | 8102521 | 79% |
| YHSigm-3 | 13350655 | 9551280 | 71.50% | 7305004 | 76% |

6. Statistical Calculation

Relative data volumes of all samples were calculated, or taken the single cell from the YH blood sample as a normal control, a ratio of relative data volumes between the single cells respectively from the T21 blood sample and the YH blood sample were calculated. The reads number of each chromosome was taken as data volume to perform statistics calculation; Table 2 showed a calculation result. Then a ratio between data volume of each chromosome of all samples and total data volume was calculated as relative data volume, shown as Table 3. A ration (Ri) of the relative data volumes between the single cells respectively from the T21 blood sample and the single cell from the YH blood sample was then calculated, in which an average value of three data volumes of YH single cell was subjected to calculation, Table 4 was a result of calculated ratio, Table 4 indicated that ratios of 21 chromosome in the sample of three T21 single cells all approximated a theoretical value of 1.5, which was obvious higher than other chromosomes, and would reflect a status of trisomy 21 correlatively.

TABLE 2
calculation of reads number in each chromosome

| | T21Sigm-1 | T21Sigm-2 | T21Sigm-3 | YHSigm-1 | YHSigm-2 | YHSigm-3 |
|---|---|---|---|---|---|---|
| total | 6940975 | 8495555 | 6290271 | 6331609 | 7828138 | 7043304 |
| chr1 | 598104 | 724964 | 522855 | 530489 | 672708 | 513667 |
| chr2 | 604736 | 755297 | 547310 | 590054 | 689275 | 706023 |
| chr3 | 509903 | 632878 | 447819 | 453631 | 568022 | 539514 |
| chr4 | 464653 | 539192 | 430795 | 437599 | 525772 | 448577 |
| chr5 | 464646 | 521476 | 409773 | 401698 | 522852 | 499346 |
| chr6 | 433874 | 563755 | 406429 | 404032 | 486509 | 388802 |
| chr7 | 391486 | 484210 | 364552 | 376660 | 436249 | 468119 |
| chr8 | 382256 | 514485 | 341599 | 331728 | 427230 | 415244 |
| chr9 | 287679 | 374924 | 254474 | 265747 | 323290 | 251093 |
| chr10 | 344276 | 440551 | 332901 | 317119 | 399017 | 324738 |
| chr11 | 343905 | 429411 | 322496 | 311699 | 387830 | 372567 |
| chr12 | 334444 | 438735 | 313062 | 320493 | 379729 | 300273 |
| chr13 | 234015 | 300237 | 217872 | 199374 | 268420 | 224625 |
| chr14 | 222854 | 262380 | 203936 | 196658 | 254132 | 230329 |
| chr15 | 207762 | 248845 | 188050 | 190304 | 240394 | 204792 |
| chr16 | 210634 | 239537 | 188140 | 192529 | 233042 | 201945 |
| chr17 | 195670 | 202183 | 167226 | 185184 | 225683 | 216575 |
| chr18 | 196839 | 238370 | 184596 | 173618 | 233921 | 234339 |
| chr19 | 137000 | 149816 | 115232 | 138937 | 152165 | 139112 |
| chr20 | 166838 | 189937 | 144621 | 150516 | 198712 | 184525 |
| chr21 | 126075 | 161879 | 115799 | 82195 | 104616 | 94828 |
| chr22 | 83326 | 82493 | 70734 | 81345 | 98570 | 84271 |
| chrX | 387112 | 514090 | 348245 | 191479 | 235763 | 222932 |
| chrY | 12093 | 10513 | 11380 | 26936 | 36523 | 35565 |

TABLE 3
relative data volume of each chromosome

| | T21Sigm-1 | T21Sigm-2 | T21Sigm-3 | YHSigm-1 | YHSigm-2 | YHSigm-3 |
|---|---|---|---|---|---|---|
| chr1 | 0.0862 | 0.0853 | 0.0831 | 0.0838 | 0.0859 | 0.0729 |
| chr2 | 0.0871 | 0.0889 | 0.0870 | 0.0932 | 0.0881 | 0.1002 |
| chr3 | 0.0735 | 0.0745 | 0.0712 | 0.0716 | 0.0726 | 0.0766 |
| chr4 | 0.0669 | 0.0635 | 0.0685 | 0.0691 | 0.0672 | 0.0637 |
| chr5 | 0.0669 | 0.0614 | 0.0651 | 0.0634 | 0.0668 | 0.0709 |
| chr6 | 0.0625 | 0.0664 | 0.0646 | 0.0638 | 0.0621 | 0.0552 |
| chr7 | 0.0564 | 0.0570 | 0.0580 | 0.0595 | 0.0557 | 0.0665 |
| chr8 | 0.0551 | 0.0606 | 0.0543 | 0.0524 | 0.0546 | 0.0590 |
| chr9 | 0.0414 | 0.0441 | 0.0405 | 0.0420 | 0.0413 | 0.0356 |
| chr10 | 0.0496 | 0.0519 | 0.0529 | 0.0501 | 0.0510 | 0.0461 |
| chr11 | 0.0495 | 0.0505 | 0.0513 | 0.0492 | 0.0495 | 0.0529 |
| chr12 | 0.0482 | 0.0516 | 0.0498 | 0.0506 | 0.0485 | 0.0426 |
| chr13 | 0.0337 | 0.0353 | 0.0346 | 0.0315 | 0.0343 | 0.0319 |
| chr14 | 0.0321 | 0.0309 | 0.0324 | 0.0311 | 0.0325 | 0.0327 |
| chr15 | 0.0299 | 0.0293 | 0.0299 | 0.0301 | 0.0307 | 0.0291 |
| chr16 | 0.0303 | 0.0282 | 0.0299 | 0.0304 | 0.0298 | 0.0287 |
| chr17 | 0.0282 | 0.0238 | 0.0266 | 0.0292 | 0.0288 | 0.0307 |
| chr18 | 0.0284 | 0.0281 | 0.0293 | 0.0274 | 0.0299 | 0.0333 |
| chr19 | 0.0197 | 0.0176 | 0.0183 | 0.0219 | 0.0194 | 0.0198 |
| chr20 | 0.0240 | 0.0224 | 0.0230 | 0.0238 | 0.0254 | 0.0262 |
| chr21 | 0.0182 | 0.0191 | 0.0184 | 0.0130 | 0.0134 | 0.0135 |
| chr22 | 0.0120 | 0.0097 | 0.0112 | 0.0128 | 0.0126 | 0.0120 |
| chrX | 0.0558 | 0.0605 | 0.0554 | 0.0302 | 0.0301 | 0.0317 |
| chrY | 0.0017 | 0.0012 | 0.0018 | 0.0043 | 0.0047 | 0.0050 |

TABLE 4
ratio of data volumes between T21 single cell sample
and YH single cell control sample.

| | T21Sigm-1 | T21Sigm-2 | T21Sigm-3 |
|---|---|---|---|
| chr1 | 1.06 | 1.05 | 1.03 |
| chr2 | 0.93 | 0.95 | 0.93 |
| chr3 | 1.00 | 1.01 | 0.97 |
| chr4 | 1.01 | 0.95 | 1.03 |
| chr5 | 1.00 | 0.91 | 0.97 |
| chr6 | 1.04 | 1.10 | 1.07 |
| chr7 | 0.93 | 0.94 | 0.96 |
| chr8 | 0.99 | 1.09 | 0.98 |
| chr9 | 1.05 | 1.11 | 1.02 |
| chr10 | 1.01 | 1.06 | 1.08 |
| chr11 | 0.98 | 1.00 | 1.01 |
| chr12 | 1.02 | 1.09 | 1.05 |
| chr13 | 1.03 | 1.08 | 1.06 |
| chr14 | 1.00 | 0.96 | 1.01 |
| chr15 | 1.00 | 0.98 | 1.00 |
| chr16 | 1.03 | 0.95 | 1.01 |
| chr17 | 0.95 | 0.80 | 0.90 |
| chr18 | 0.94 | 0.93 | 0.97 |
| chr19 | 0.97 | 0.87 | 0.90 |
| chr20 | 0.95 | 0.89 | 0.91 |
| chr21 | 1.37 | 1.43 | 1.39 |
| chr22 | 0.96 | 0.78 | 0.90 |
| chrX | 1.82 | 1.97 | 1.81 |
| chrY | 0.37 | 0.26 | 0.39 |

7. Statistical Test of the Obtained Calculated Data, and Determination Whether the Chromosome has an Abnormality

The relative data volume ratio (Ri) obtained above was subjected to a Student's t-test. In short, the relative data volume ratio of each chromosome T21Sigm-1, T21Sigm-2, and T21Sigm-3 was subjected to calculating a mean value and a standard deviation, and based on formula

$$z - score = \frac{{R}_{i} - mean}{sd}$$

Z-score of each chromosome was calculated, Table 5 was calculated Z-score value of each chromosome. According to the normal distribution theory, in a case of −3<Z-score value<3, the chromosome was determined to be normal; otherwise, in a case of Z-score value exceeding the above range, the chromosome was determined having an abnormality. Since the gender was different between the T21 sample (female) and the YH sample (male), a ratio of sex chromosome was not subjected to Z-score calculation. The result showed that the Z-score values of chromosome 21 from three T21 single cell samples were all more than 3, having a significant difference, which could be determined as Trisomy 21.

TABLE 5
Z-score value obtained from calculating the relative
data volume ratio of each autosome

| | T21Sigm-1 | T21Sigm-2 | T21Sigm-3 |
|---|---|---|---|
| chr1 | 0.62 | 0.40 | 0.16 |
| chr2 | −0.90 | −0.37 | −0.80 |
| chr3 | −0.14 | 0.09 | −0.43 |
| chr4 | −0.05 | −0.35 | 0.18 |
| chr5 | −0.15 | −0.64 | −0.39 |
| chr6 | 0.30 | 0.74 | 0.60 |
| chr7 | −0.87 | −0.42 | −0.50 |
| chr8 | −0.18 | 0.69 | −0.29 |
| chr9 | 0.41 | 0.84 | 0.11 |
| chr10 | 0.00 | 0.42 | 0.67 |
| chr11 | −0.34 | 0.00 | 0.04 |
| chr12 | 0.13 | 0.70 | 0.44 |
| chr13 | 0.25 | 0.61 | 0.50 |
| chr14 | −0.12 | −0.29 | −0.01 |
| chr15 | −0.13 | −0.17 | −0.12 |
| chr16 | 0.17 | −0.35 | 0.01 |
| chr17 | −0.65 | −1.45 | −1.10 |
| chr18 | −0.83 | −0.54 | −0.40 |
| chr19 | −0.42 | −0.97 | −1.06 |
| chr20 | −0.63 | −0.83 | −0.96 |
| chr21 | 4.06 | 3.21 | 3.72 |
| chr22 | −0.53 | −1.63 | −1.06 |

8. calculation of an average value and a standard deviation with relative data volumes of three normal control YH single cell samples (YHSigm-1, YHSigm-2, YHSigm-3) to T21Sigm-1, and Z-score value calculation with the relative data volume of T21 single cell sample to be tested by this model, shown in Table 6. According to the normal distribution theory, in a case of −3<Z-score value<3, the chromosome was determined to be normal; otherwise, in a case of Z-score value exceeding the above range, the chromosome was determined having an abnormality. For sex chromosome X, in the present example, the T21 sample to be tested was determined having an extra X chromosome comparing with the control sample based on the Z-score value, since the control sample YH was from a male, the T21 sample to be tested could be determined to be a female. The Z-score values of chromosome 21 from three T21 single cell samples were all greater than 3, having a significant difference, which could be determined as Trisomy 21. Since the genders were different between the T21 sample (female) and the YH sample (male), a ratio of sex chromosome was not subjected to Z-score value calculation.

TABLE 6
Z-score value obtained from calculating the relative
data volume of each autosome

| | T21Sigm-1 | T21Sigm-2 | T21Sigm-3 |
|---|---|---|---|
| chr1 | 0.76 | 0.64 | 0.32 |
| chr2 | −1.10 | −0.80 | −1.11 |
| chr3 | −0.05 | 0.34 | −0.91 |
| chr4 | 0.10 | −1.16 | 0.67 |
| chr5 | −0.03 | −1.52 | −0.51 |
| chr6 | 0.46 | 1.31 | 0.93 |
| chr7 | −0.76 | −0.65 | −0.48 |
| chr8 | −0.07 | 1.57 | −0.30 |
| chr9 | 0.52 | 1.29 | 0.23 |
| chr10 | 0.21 | 1.08 | 1.49 |
| chr11 | −0.50 | −0.01 | 0.35 |
| chr12 | 0.23 | 1.06 | 0.61 |
| chr13 | 0.77 | 1.84 | 1.37 |
| chr14 | 0.04 | −1.34 | 0.39 |
| chr15 | −0.02 | −0.80 | −0.06 |
| chr16 | 0.83 | −1.62 | 0.33 |
| chr17 | −1.41 | −5.76 | −3.00 |
| chr18 | −0.62 | −0.73 | −0.29 |
| chr19 | −0.47 | −2.01 | −1.51 |
| chr20 | −0.88 | −2.24 | −1.72 |
| chr21 | 19.24 | 22.74 | 20.20 |
| chr22 | −1.02 | −6.07 | −2.69 |
| chrX | 29.46 | 35.02 | 28.98 |

Reference throughout this specification to “an embodiment,” “some embodiments,” “one embodiment”, “another example,” “an example,” “a specific example,” or “some examples,” means that a particular feature, structure, material, or characteristic described in connection with the embodiment or example is included in at least one embodiment or example of the present disclosure. Thus, the appearances of the phrases such as “in some embodiments,” “in one embodiment”, “in an embodiment”, “in another example,” “in an example,” “in a specific example,” or “in some examples,” in various places throughout this specification are not necessarily referring to the same embodiment or example of the present disclosure. Furthermore, the particular features, structures, materials, or characteristics may be combined in any suitable manner in one or more embodiments or examples.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

1. A method of determining a chromosome aneuploidy of a single cell, comprising:
sequencing a whole genome of the single cell to obtain a first sequencing result;
counting the total number of sequencing data which can be aligned to a reference genome in the first sequencing result, to obtain a value L;
counting the number of sequencing data which can be aligned to a first chromosome in the reference genome in the first sequencing result, to obtain a value M;
determining a first parameter based on the value L and the value M;
determining whether the single cell has an aneuploidy with respect to the first chromosome, based on a difference between the first parameter and a preset control parameter.

2. The method of claim 1 further comprising a step of isolating the single cell from a biological sample;
wherein the biological sample is at least one selected from a group consisting of blood, urine, saliva, tissue, germ cell, blastomere, and embryo;
wherein isolating the single cell from the biological sample is performed by at least one from a group consisting of dilution, mouth-controlled pipette isolation, micromanipulation, flow cytometry isolation and microfluidic;
wherein the micromanipulation is micro-dissection.

3.-5. (canceled)

6. The method of claim 1, wherein sequencing the whole genome of the single cell further comprises:
amplifying the whole genome of the single cell to obtain an amplified whole genome;
constructing a whole genome sequencing-library using the amplified whole genome; and
sequencing the whole genome sequencing-library to obtain a plurality of sequencing data, wherein the plurality of sequencing data constitute the first sequencing result.

7. The method of claim 6 further comprising a step of lysing the single cell to release the whole genome of the single cell;
wherein lysing the single cell to release the whole genome of the single cell is performed using an alkaline lysis buffer.

8. (canceled)

9. The method of claim 6, wherein amplifying the whole genome is performed using a PCR-based whole genome amplification method;
wherein the PCR-based whole genome amplification method is OmniPlex WGA.

10. (canceled)

11. The method of claim 6, wherein sequencing the whole genome sequencing-library is performed using at least one selected from a group consisting of Hiseq2000, SOLiD, Roche 454, and single-molecule sequencing apparatus.

12. The method of claim 6, wherein the plurality of sequencing data has an average length of about 50 bp.

13. The method of claim 1, wherein the first chromosome is at least one selected from human chromosome 21, chromosome 18, chromosome 13, chromosome X and chromosome Y.

14. The method of claim 1, wherein the first parameter is a ratio M/L of the value M to the value L;
wherein the preset control parameter is obtained by the steps of:
sequencing a whole genome of a control single cell to obtain a second sequencing result, wherein the whole genome of the control single cell derives from a sample without the chromosome aneuploidy;
counting the total number of sequencing data which can be aligned to a reference genome in sequencing data of the second sequencing result, to obtain a value L′;
counting the number of sequencing data which can be aligned to the first chromosome of the reference genome in the second sequencing result, to obtain a value M′; and
determining a ratio M′/L′ of the value M′ to the value L′, to obtain the preset control parameter.

15. (canceled)

16. The method of claim 14, wherein:
in the case of a ratio of the first parameter to the preset control parameter exceeding a first threshold, the number of the first chromosome of the single cell is determined to be 3;
in the case of the ratio of the first parameter to the preset control parameter falling below a second threshold, the number of the first chromosome of the single cell is determined to be 1; and
in the case of the ratio of the first parameter to the preset control parameter being between the first threshold and the second threshold, the number of the first chromosome of the single cell is determined to be 2.

17. The method of claim 1 further comprising a step of subjecting the ratio of the first parameter to the preset control parameter to a Student's t-test, to obtain a Student's t-test value of the first chromosome.

18. The method of claim 1 further comprising a step of subjecting the first parameter and the preset control parameter to a Student's t-test respectively, to obtain a Student's t-test value of the first chromosome.

19. A system for determining a chromosome aneuploidy of a single cell, comprising:
a whole genome sequencing apparatus, for sequencing a whole genome of the single cell to obtain a first sequencing result;
a sequencing result analyzing apparatus, connected to the whole genome sequencing apparatus, configured to receive the first sequencing result from the whole genome sequencing apparatus and to perform following steps:
counting the total number of sequencing data which can be aligned to a reference genome in sequencing data of the first sequencing result, to obtain a value L;
counting the number of sequencing data which can be aligned to a first chromosome in the reference genome in the first sequencing result, to obtain a value M;
determining a first parameter based on the value L and the value M;
determining whether the single cell has an aneuploidy with respect to the first chromosome, based on a difference between the first parameter and a preset control parameter.

20. The system of claim 19 further comprising a whole genome sequencing-library constructing apparatus, wherein the whole genome sequencing-library constructing apparatus provides the whole genome sequencing-library for sequencing to the whole genome sequencing apparatus;
wherein the whole genome sequencing-library constructing apparatus further comprises:
a single cell isolating unit, for isolating the single cell from a biological sample;
a single cell lysing unit, for receiving an isolated single cell and lysing the single cell, to release the whole genome of the single cell;
a whole genome amplifying unit, connected to the single cell lysing unit, for receiving the whole genome of the single cell and amplifying the whole genome of the single cell; and
a sequencing-library constructing unit, for receiving an amplified whole genome, and constructing the whole genome sequencing-library using the amplified whole genome.

21. The system of claim 19, wherein the single cell isolating unit comprises at least one apparatus suitable for performing following operations selected from a group consisting of dilution, mouth-controlled pipette isolation, micromanipulation, flow cytometry isolation, and microfluidic;
wherein the micromanipulation is micro-dissection;
wherein the single cell lysing unit comprises an apparatus suitable for lysing the single cell using an alkaline lysis buffer.

22.-23. (canceled)

24. The system of claim 19, wherein the whole genome amplifying unit comprises an apparatus suitable for amplifying the whole genome using a PCR-based whole genome amplification method;
wherein the PCR-based whole genome amplification method is OmniPlex WGA.

25. (canceled)

26. The system of claim 20, wherein the whole genome sequencing apparatus comprises at least one selected from a group consisting of Hiseq2000, SOLiD, Roche 454, and single-molecule sequencing apparatus.

27. The system of claim 20, wherein the sequencing result analyzing apparatus further comprises a sequence aligning unit, for aligning the first sequencing result with known genomic sequence information, to obtain all sequencing data which can be aligned to the reference genome, and to obtain sequencing data deriving from the first chromosome.

28. The system of claim 20, wherein the sequencing result analyzing apparatus further comprises a Student's t-test unit, for subjecting a ratio of the first parameter to the preset control parameter to a Student's t-test, to obtain a Student's t-test value of the first chromosome.

29. The system of claim 19, wherein the first parameter is a ratio M/L of the value M to the value L;
wherein the system is configured to obtain the preset control parameter by:
sequencing a whole genome of a control single cell to obtain a second sequencing result, wherein the whole genome of the control single derives from a sample without the chromosome aneuploidy;
counting the total number of a sequencing data which can be aligned to a reference genome in the sequencing data of the second sequencing result, to obtain a value L′;
counting the number of a sequencing data which can be aligned to the first chromosome of the reference genome in the second sequencing result, to obtain a value M′; and
determining a ratio M′/L′ of the value M′ to the value L′, to obtain the preset control parameter.
